(19) United States
(12) Patent Application Publication
(10) Pub. No.: US 2005/0170387 A1
(43) Pub. Date: Aug. 4, 2005

(54) Gout related genetic locus

(76) Inventors: Ying-Chin Ko, Kaohsiung (TW); Li Shu-Chuan Cheng, San Marino, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/013,492

(22) Filed: Dec. 15, 2004

Related U.S. Application Data
(60) Provisional application No. 60/530,271, filed on Dec. 16, 2003.

(51) Int. Cl. C12Q 1/68; C07H 21/04; C12N 9/06

(52) U.S. Cl. 435/6; 435/69.1; 435/320.1; 435/325; 435/191; 536/23.2

(57) ABSTRACT

The present invention relates to a gout related gene locus located in the genomic region of about 90 cM to about 150 cM on chromosome 4, which region is flanked by genome markers D4S2361 and D4S1644. The genomic region can be used in a haplotype assay of the genome markers found in the region to determine whether a family member of a gout subject has the propensity to become inflicted with gout. The same genome markers can be used in a haplotype assay to determine whether a family member of a hyperuricemia subject has the propensity to become inflicted with hyperuricemia.

This application claims the benefit of U.S. Provisional Application No. 60/530,271, filed Dec. 16, 2003, the contents of which are incorporated herein in their entireties.

### FIELD OF INVENTION

This invention relates generally to a gout related genomic region.

### BACKGROUND OF THE INVENTION

Gout is a disorder of uric acid metabolism. It is of particular interest in the Pacific Austronesian population because the population, including Taiwanese aborigines, has a remarkably high prevalence of hyperuricemia and gout, suggesting a founder effect across the Pacific.

Gout (MIM 138900 (Mendelian Inheritance in Man, a database of human genes and genetic disorders of Johns Hopkins University)) is characterized by elevated serum-urate levels and recurrent attacks of intra-articular crystal deposition of monosodium urate monohydrate. Clinical manifestations include recurrent painful attacks of acute inflammatory arthritis, tophi, uric acid urolithiasis, renal impairment and, eventually, renal failure.

Uric acid is produced within all mammalian cells as the product of purine degradation. Homeostasis of uric acid depends on the balance between cellular production and renal clearance. Hyperuricemia develops as a result of overproduction or decreased renal excretion of uric acid. The incidence of acute gout is about 5% each year among patients with hyperuricemia and a serum-urate concentration of 9.0 mg/dl (Campion et al. (1987) Am. J. Med. 82: 421-426).

Gout has genetic components and is complicated by environmental factors such as diet and alcohol intake, and by age and sex, e.g. menopause in women. Gout related genetic correlations are based on rare forms of Mendelian disorders, e.g. hypoxanthine guanine phosphoribosyltranserase (HPRT) for X-linked gout, Autosomal Dominant Medullary Cystic Disease on chromosome 1q21, Familial Juvenile Hyperuricemic Nephropathy on chromosome 16, Uric Acid Nephrolithiasis on chromosome 10, or genes for uric acid transport in kidney (see OMIM (Online Mendelian Inheritance in Man) 138900 gout).

A series of studies on indigenous groups from Polynesia (Prigent et al. (1992) Med. Trop. 52: 63-6; Jackson et al. (1981) J. Chronic Dis. 34: 65-76; Prior et al. (1987) Br. Med. J. 295: 457-461), Melanesia (Prior (1981) Semin. Arthirtis Rheum. 11: 213-229), Micronesia (Zimmet et al. (1978) Br. Med. J. 1: 1237-1239), Indonesia (Darmawan et al. (1992) J. Rheumatol. 19: 1595-1599) and Taiwan (Chang et al. (1997) J. Rheumatol. 24: 1364-1369) show significantly higher uric acid levels than that found in the white populations. Linguistic, archaeological, and genetic evidence suggest that the insular populations across the Pacific region, including Polynesians, Micronesians, Melanesians, and Taiwan aborigines, are part of an Austronesian population (Bellwood (1991) Sci. Am. 70: 70-75; Diamond (2000) Nature 403: 709-710; Gray and Jordan (2000) Nature 405: 1052-1055; Chang et al. (2002) J. Hum. Genet. 47: 60-5; Diamond and Bellwood (2003) Science 25: 597-603). This genetic relationship and the high morbidity of gout in these Austronesian populations suggest a possible founder effect of gout susceptibility.

A genetic component of gout has been suggested but there have been no other known report of significant finding from linkage studies, except some rare Mendelian syndromes such as Autosomal Dominant Medullary Cystic Disease or Familial Juvenile Hyperuricemic Nephropathy as noted above. Studies failed to identify genes for the complex gout related traits from systematic genome search. This may be attributed to the heterogeneity of the disease and inadequate power on sample size, sample structure, or statistical methods.

### SUMMARY OF THE INVENTION

The present invention relates to a DNA segment from chromosome 4 flanked by genome markers D4S2361 and D4S1644. In one embodiment, the present invention relates to a segment of DNA located in the genomic region of 90 cM to 150 cM on chromosome 4. In yet another embodiment, the invention relates to a gout gene locus located on chromosome 4q25 at the genome marker D4S2623, which is located at 114 cM on chromosome 4. In addition, the invention relates to a method of determining the propensity for gout in the family members of a gout subject using haplotype analysis of genome markers within the genomic region of 90 cM to 150 cM on chromosome 4. The invention also provides a method of determining the propensity for hyperuricemia in the family members of a hyperuricemia subject using the same haplotype analysis of genome markers within the genomic region of 90 cM to 150 cM on chromosome 4.

### BRIEF DESCRIPTION OF THE DRAWINGS

FIG. 1 shows the NPL Z score obtained from MERLIN software for all 22 autosomes. The highest NPL Z score (3.58) among the 22 autosomes scanned is located at 114 cM on chromosome 4.

FIG. 2 shows the LOD scores from multipoint linkage analysis, by conditional-logistic model, on chromosome 4 with a peak location at marker D4S2623, also located at 114 cM.

### DETAILED DESCRIPTION OF THE INVENTION

The present invention localizes the gout disease susceptibility locus (gout related locus) from a genome search. The conducted study has great power from both the multiplexity of the pedigree samples and unique nature of an isolated population, the aborigines in Taiwan. The latter, presumably, is more homogeneous in genetic and common environmental effects (Wright et al. (1999) Nature Genetics 23: 397-403). The gout related locus is found on chromosome 4 between about 90 cM to about 150 cM or most likely at about 114 cM.

Definitions

As used herein, the term “isolated” refers to being separated from other nucleic acid molecules which are present in the natural source of nucleic acids. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

As used herein, the term “DNA segment” refers to a piece of deoxyribonucleic acid molecule (DNA) or cDNA molecule which is a synthetic DNA transcribed from a specific RNA through the reaction of the enzyme reverse transcriptase.

As used herein, the term “genomic region” refers to a portion or section of the genome.

As used herein, the term “flanked” refers to situated in between (and including) genome markers.

As used herein, the term “genome markers” refers to a segment of DNA with an identifiable physical location on a chromosome whose inheritance can be followed. A marker can be a gene, or it can be some section of DNA with no known function.

As used herein, the term “cM” refers to centimorgan on a Marshfield Genetic Map (Center for Medical Genetics, Marshfield Medical Research Foundation).

As used herein, the term “amplifying” refers to replication of a gene or DNA sequence, such as in a polymerase chain reaction.

As used herein, the term “gout subject” refers to a person affected with gout.

As used herein, the term “family member” (or “family members”) refers to one or more members of a family, including immediate and extended family members whose blood relationship can be traced.

As used herein, the term “haplotype” refers to a set of alleles of a group of closely linked genes which is usually inherited as a unit.

As used herein, the term “propensity for gout” refers to having the tendency or likelihood to become inflicted with gout.

As used herein, the term “identical” refers to being exactly the same.

As used herein, the term “propensity for hyperuricemia” refers to having the tendency or likelihood to become inflicted with hyperuricemia, a disease of having an excess of uric acid or urate in the blood.

The Gout Related Locus

The present invention provides the findings of a genome-wide linkage study on 21 multiplex gouty pedigrees from an isolated highland aboriginal tribe in Taiwan. This population is presumably more homogeneous and thus provides a better power to illustrate genetic effects than other populations (Wright et al. (1999) Nature Genetics 23: 397-403). From the observation of familial clustering, early onset of gout, and clinically severe manifestations, it was hypothesized that one or more major genes plays a role in this disorder.

In the present invention, a genomic region related to gout is identified by genotyping and various statistical analysis using 382 random short tandem repeat polymorphic markers spread across 22 autosomes. These markers have multiple alleles and high heterozygosities. The identified region is marked by sequential genome markers D4S2361, D4S1647, D4S2623, TAGA006, D4S2394, and D4S1644, located at an avera distance of 10 cM apart. There is a highly significant linkage for gout at genome marker D4S2623 on chromosome 4q25, at about 114 cM on chromosome 4 (p=0.0002 by NPLₐₗₗ; empirical P=0.0006; LOD score=4.3, p=4.4×10⁻⁶ by logistic regressions, when alcohol consumption was included as a covariate in the model, the LOD score increased to 5.66 (p=1.3×10⁻⁶)). Quantitative traits including serum uric acid and creatinine also showed a moderate linkage to this region.

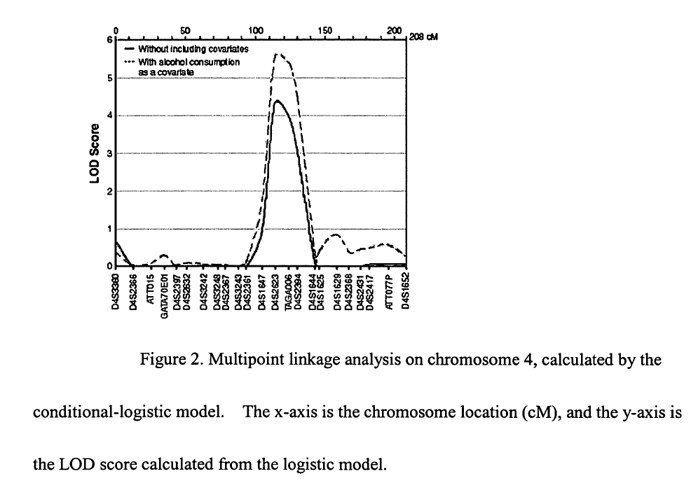

The invention thus provides a DNA segment flanked by genome markers D4S2361 and D4S1644, and within this region, there are other genome markers, i.e. D4S1647, D4S2623, TAGA006, D4S2394, as set forth in FIG. 2. The invention further provides a DNA segment flanked by genome markers D4S1647 and D4S2394. Also provided is the DNA segment flanked by genome markers D4S2623 and TAGA006. The invention further provides the genomic region flanked by genome markers D4S2623 and D4S1647. Also provided is the DNA segment flanked by genome markers D4S2623 and D4S2361. The invention also provides the DNA segment flanked by genome markers D4S2623 and D4S2394. Additionally, the invention provides the DNA segment flanked by genome markers D4S2623 and D4S1644.

In another embodiment, the invention provides a DNA segment located in the genomic region of about 90 cM to about 150 cM on chromosome 4. This is based on the relative position of the genome markers spaced at an average distance of 10 cM apart with D4S2623 at about 114 cM. The invention further describes a DNA segment located in the genomic region of about 100 cM to about 140 cM on chromosome 4. Alternatively, the invention provides a DNA segment located in the genomic region of about 110 cM to about 130 cM on the same chromosome.

The invention also provides a method of determining the propensity for gout, for example in the family members of a gout subject. In one embodiment, the DNA segment flanked by D4S2361 and D4S1644 of a gout subject and his/her family member are genotyped. Then, the haplotype of genome marker D4S2623 and at least one other genome marker selected from D4S2361, D4S1647, TAGA006, D4S2394, and D4S1644 is determined. Subsequently, the haplotype of the gout subject and that of the family member are compared, and the family member is identified to have the propensity for gout if the haplotypes are identical.

In another embodiment, the DNA segment flanked by D4S1647 and D4S2394 of a gout subject and his/her family member are genotyped. Then, the haplotype of genome marker D4S2623 and at least one other genome marker selected from D4S1647, TAGA006, and D4S2394 is determined. If the haplotype of the family member and the gout subject are identical, then the family member is identified to have the propensity for gout.

The invention also provides for a method of determining the propensity for gout, for example in the family members of the gout subject by amplifying the DNA segment flanked by D4S2623 and TAGA006. As with the prior embodiment, the haplotypes of D4S2623 and TAGA006 of the gout subject and his/her family member are determined and compared. The family member is identified to have the propensity for gout if the haplotypes are identical.

Moreover, the invention relates to a method of determining the propensity for hyperuricemia in the family members of a hyperuricemia subject by the same haplotype analysis using the genome marker D4S2623 and at least one other genome marker selected from D4S2361, D4S1647, TAGA006, D4S2394, and D4S1644.

Implication of the Gout Locus

Interestingly, the strongest signal, marker D4S2623, located at 114 cM of the Marshfield genetic map is about 1.4 cM apart from a longevity locus (Puca et al. (2001) Proc. Nat. Acad. Sci. USA 98: 10505-10508). The coincident mapping of a gout candidate gene and a longevity gene implies either that this region may harbor two separate susceptibility genes for gout and longevity, or that a common gene in this region is responsible for both traits. The identification of a gout susceptible gene in the 4q25 region may shed light on the pathogenesis of gout and possibly the mechanisms of longevity.

There is also a positive correlation between lifespan and the concentration of uric acid, suggesting a role of uric acid in longevity. Free radicals have long been implicated in aging theory (Harman D. (1957) J. Gerontol 2: 298-300), where aging may result from cumulative oxidative damage in cells, and increased resistance to oxidative damage may extend the life span. Uric acid scavenges potentially harmful reactive oxygen species and is thought to be a primary anti-oxidant in humans because of its high levels in plasma as compared to other anti-oxidants (Ames B. N. et al. (1981) Proc. Natl. Acad. Sci. USA 78: 6858-6862). The highly significant positive correlation between lifespan and the concentration of urate in serum and brain among mammalian species (Cutler R. G. (1984) Proc. Natl. Acad. Sci. USA 3: 321-48) also implicates the role of uric acid in longevity. In addition, unlike in most mammals where uric acid is degraded by urate oxidase (uricase) to allantoin and excreted in the urine, in human and the great apes, uric acid levels are higher (>2 mg/dL) resulting from the uricase mutations that occurred during early hominoid evolution about 5 to 20 million years ago (Ames B. N. et al. (1981) Proc. Natl. Acad. Sci. USA 78: 6858-6862; Cutler R. G. (1984) Proc. Natl. Acad. Sci. USA 3: 321-48). This evolutionary event may partially account for the longer lifespan of human and the great apes than that of most other primates (Ames B. N. et al. (1981) Proc. Natl. Acad. Sci. USA 78: 6858-6862; Cutler R. G. (1984) Proc. Natl. Acad. Sci. USA 3: 321-48).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

Further, at the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement

It must be noted that, as used herein and in the appended claims, the singular forms “a,” “or,” and “the” include plural referents unless the context clearly dictates otherwise.

The following examples further illustrate the invention. They are merely illustrative of the invention and disclose various beneficial properties of certain embodiments of the invention. The examples should not be construed as limiting the invention.

### EXAMPLES

The practice of the present invention will employ, unless otherwise indicated, conventional techniques of genetics study and statistical analysis, which are within the skill of the art. Such techniques are explained fully in the literature.

The following examples illustrate the organization of study subjects with and without gout and with and without hyperuricemia, and they also illustrate the analysis of their genotypes through genetics statistical software to ascertain the locus on the chromosome related to gout.

### Example 1

### Assemblage of Study Subjects

For the study of gout related genes, efforts were made to gather study subjects with and without the gout disease. Study subjects were from an aboriginal tribe in Taiwan. The ascertainment of the gout probands (subjects) was initially made through a community public health survey conducted in the local Health Stations in Taiwan, with the aim of health education and prevention. Through the health survey, general health history and specific conditions, including gout and hypertension, of middle-aged and elderly people were collected. The diagnosis of gout probands was confirmed by a rheumatologist based on the criteria set out in Wallace (Wallace et al. (1977) Arthritis Rheum. 20:895-900). Family structure and family history of each proband were obtained in the survey. All affected members reported by the probands were confirmed by the rheumatologist. In total, we recruited 154 individuals belonging to 21 pedigrees. Out of the 154 participants, there were 92 confirmed gout subjects and 62 subjects not affected with gout. Out of the 62 unaffected subjects, there were 29 subjects with hyperuricemia (having uric acid index of greater than or equal to 7.5) and 33 normal subjects. Blood was drawn by trained medical technologists. Informed consent from each study subject was obtained before the study.

### Example 2

### Comparison of Characteristics of Gout Affected and Unaffected Subjects

Data on specific health history and conditions were collected for both the subjects affected with gout and the unaffected subjects. There was no significant difference in age between the group with gout and the unaffected group (Table 1). More subjects with gout consumed alcohol on a regular basis (at least twice a week) than the unaffected subjects. However, for the affected subjects, the starting age and duration of drinking and liver function measurements (GOT [glutamic-oxaloacetic transaminase], GPT [glutamic-pyruvic transaminase]) do not differ from the unaffected subjects. The affected group also had higher blood pressure, higher levels of serum uric acid and cholesterol than those of the unaffected group. No significant difference was found in BMI (body mass index) between the affected and the unaffected groups.

TABLE 1
Characteristics of Subjects with Gout and Unaffected Subjects from
21 Multiplex Aboriginal Families in Taiwan

| | Gout (91) | Non-gout (63) | |
|---|---|---|---|
| Age, yrs | 47.3 ± 14.9 | 46.9 ± 15.0 | P = 0.90 |
| Alcohol-drinking | | | |
| Regularly | 56 | 27 | P = 0.02 |
| Rarely | 35 | 36 | |
| Alcohol-drinking | 21.87 ± 6.5 | 23.2 ± 7.3 | P = 0.57 |
| starting Age, yrs | | | |
| Alcohol-drinking no. of | 20.2 ± 10.2 | 23.3 ± 17.0 | P = 0.46 |
| years | | | |
| BMI, kg/m² | 25.4 ± 4.1 | 25.5 ± 4.2 | P = 0.99 |
| Uric acid, mg/dL | 9.5 ± 2.4 | 7.6 ± 1.8 | P < 0.001 |
| Blood pressure, mmHg | | | |
| Systolic | 136.4 ± 23.2 | 127.7 ± 24.2 | P = 0.028 |
| Diastolic | 87.9 ± 15.5 | 82.3 ± 15.1 | P = 0.029 |
| Triglyceride, mg/dL | 265.8 ± 240.7 | 207.8 ± 120.3 | P = 0.053 |
| Cholesterol, mg/dL | 190.3 ± 45.3 | 176.6 ± 27.1 | P = 0.022 |
| Liver function (IU/L) | | | |
| GOT, IU/L | 30.9 ± 27.2 | 27.7 ± 15.4 | P = 0.40 |
| GPT, IU/L | 29.6 ± 20.4 | 27.4 ± 24.9 | P = 0.54 |

### Example 3

### Genotyping

Genetic analysis was carried out with respect to the 154 gout study subjects. Genomic DNA was prepared from blood with the PureGene DNA Isolation Kit (Gentra System, Minneapolis). Genotyping was provided by Mammalian Genotyping Service of Marshfield (Center for Medical Genetics, Marshfield Medical Research Foundation), using PCR (polymerase chain reaction) and fluorescent labeled primers, and the PCR product was analyzed by Gel electrophoresis. The samples were genotyped using the newer screening set, Weber Screening Set 13 (see the Center for Medical Genetics Website) which has more accurate allele calling and less error or missing typing as a consequence of using a higher quality of tri and tetra-nucleotide STRPs (short tandem repeat polymorphisms) with a more accurate map and spacing (Ghebranious et al. (2003) BMC Genomics 24: 6 2003). Three hundred and eighty-two (382) autosomal markers were used in this study with an averaged 9.3 cM spacing (range 0.3-18 cM) and 0.69 heterozygosity.

As an error check on the genotyping, PedCheck program (O'Connell and Weeks (1998) Am. J. Hum. Genet. 63:259-266) and the MERLIN program (Abecasis et al. (2002) Nature Genetics 3:97-10 1) were applied to check any inconsistent Mendelian inheritance, non-paternity, or any genotyping error. Any inconsistency was zeroed out to avoid bias. Three individuals were removed from the analysis because of many genotyping errors found in those samples. Gene frequencies were estimated by allele counting in all genotyped individuals and were automatically calculated by MERLIN. The results were not different when other strategies were used to calculate the allele frequency such as maximum likelihood estimate.

### Example 4

### Statistical Analysis

Various statistical analyses were carried out to determine and confirm gout related genetic linkage. Among the 21 families, one extended pedigree was too big to fit the software programs used, therefore this pedigree was divided into five families (giving rise to a total of 25 pedigrees) for nonparametric linkage analysis (MERLIN software (Multimpoint Engine for Rapid Likelihood Inference) (Abecasis et al. (2002) Nature Genetics 3:97-101)), or into three families (giving rise to a total of 23 pedigrees) for conditional-logistic model analysis (LODPAL in S.A.G.E. software (Statistical Analysis for Genetic Epidemiology) (Olson (1999) Am. J. Hum. Genet. 65:1760-1769)). Among the 23 pedigrees that were analyzed by the logistic model, there were a total of 66 affected sibpairs, 30 affected parent-child pairs, four affected half sibpairs, 14 grandparent-children pairs, 61 affected avuncular pairs, and 29 affected cousin pairs.

To examine the false positive rate in the genome scan, ten thousand (10,000) multipoint simulations were performed under the null hypothesis of no linkage or association to the phenotype by using the MERLIN program. The simulation generated random marker data sets that keep the original data including family structure, phenotypes, marker informativeness, map distance and missing data patterns through gene dropping procedures (Abecasis et al. (2002) Nature Genetics 3:97-101). Empirical p value was determined by the number of replicates that exceed the observed Z score, divided by total replicates (10,000).

### Nonparametric Linkage Analysis

Multipoint nonparametric linkage (NPL) analysis was performed on the error-checked genotype data on 382 autosomal markers. For discrete traits, nonparametric linkage (NPL) analysis was used (Kruglyak et al. (1996) Am. J. Hum. Genet. 58: 1347-1363; Whittemore and Halpern (1994) Biometrics 50:118-27) employing the MERLIN program (Abecasis et al. (2002) Nature Genetics 3:97-101). This method calculates inheritance distribution for sets of affected pairs and then uses a score function to determine the significance of linkage. The analysis was based on identical by descent (IBD) sharing among affected relative pairs using all marker information in each chromosome to screen all 22 chromosomes linkage results. NPLₐₗₗ was used, which estimated identical by descent allele (IBD) sharing among all affected members and was averaged over all possible inheritance patterns, normalized, and weighted across pedigrees.

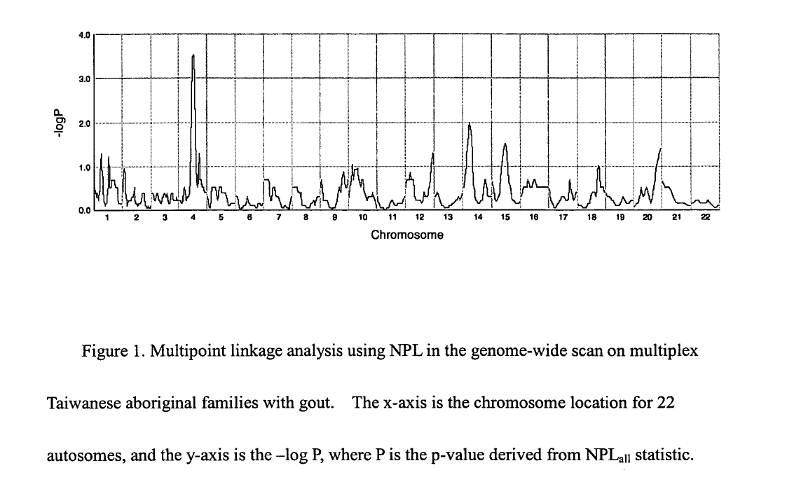

FIG. 1 shows the NPL Z score obtained from MERLIN software for all 22 chromosomes. The highest peak is in chromosome 4 with −log P 3.70 (NPL Z score=3.58). The highest NPL Z score (3.58) among the 22 chromosomes scanned was located at 114 cM on chromosome 4 with a p-value of 0.0002 (FIG. 1). The empirical p-value was 0.0006.

### Conditional-logistic Model Analysis

An alternative approach, the conditional-logistic model, was employed to further confirm the findings on chromosome 4. (Some of the pedigrees were complicated and their information may not be fully utilized by the NPL statistics.) The multipoint analysis using conditional-logistic model (Olson (1999) Am. J. Hum. Genet. 65: 1760-1769). implemented in the S.A.G.E. package was applied. This approach is parameterized in terms of the allele-sharing-specific relative risks and may capture more information than the NPL method. This method estimates parameter βs based on relative risk λi for a pair of relatives that shares i allele(s) identical by descent (IBD), where λi=e^{βi}. Multipoint IBD estimates were obtained using the GENIBD program in S.A.G.E. The one-parameter model was used along with the default value that constrains the relative risks, λ₂=3.634λ₁ −2.634, without assuming any mode of inheritance. Likelihood ratio statistics (LRS) was computed by multiplying the LOD score by 4.6. The p-value for one-parameter model was derived from the LRS distribution with 50:50 mixture of a point mass at 0 and a 1-degree of freedom chi-square distribution (1−dfχ²). When alcohol consumption was included in the model, the LRS distribution was a 50:50 mixture of a χ² with 1 df and a χ² with 2 df (Goddard et al. (2001) Am. J. Hum. Genet. 68: 1197-1206). The significance of the covariate was determined by the difference between the LRS with the covariate and the LRS without the covariate.

Based on the logistic model, the maximal LOD score for chromosome 4 was also located at 114 cM with a LOD score of 4.3, with a peak location at marker D4S2623 (p-value=0.0000044, Table 2 and FIG. 2). Because alcohol consumption plays a role in gout development, alcohol drinking was included as a covariate in the logistic model. The LOD score increased to 5.66 and the overall p-value for the linkage was 0.0000013 based on a mixture of 1 and 2 degree-freedom (1−df, 2−df) distributions. The alcohol consumption was significant with a p-value of 0.012 (χ²=6.30, 1 degree of freedom). The corresponding marker at this peak location was D4S2623. The LOD score from single point analysis for this marker was 6.37 under a one-parameter model (data not shown). The 1-LOD score support intervals were from 107 cM to 129 cM. The estimate of β1 from one-parameter model with the drinking covariate was 0.99, converting to λ₁=2.68, and λ₂=7.10. This means the relative risk for an individual who shares one allele IBD with an affected relative is almost 3; the relative risk for the person sharing two allele IBD with an affected relative is over 7.

TABLE 2
Linkage Results of Gout and Its Quantitative Traits on Chromosome 4
by Different Statistical Methods

| | | | Position | |
|---|---|---|---|---|
| Trait | LCD Score | p-value | (cM) | Method |
| Gout | 4.29 | 0.0000044 | 114 | Conditional-logistic |
| | | | | model |
| Gout with | 5.66 | 0.0000013 | 114 | Conditional-logistic |
| covariate* | | | | model |
| Gout | 3.58 | 0.0002** | 114 | NPL*** |
| Uric Acid | 1.11 | 0.012** | 114 | Deviate**** |
| Creatinine | 1.47 | 0.005** | 114 | Deviate |

*Alcohol consumption was added in a binary form as a covariate in the one-parameter logistic model.
**Empirical P values, based on 10,000 simulations and calculated by MERLIN, were 0.0006 for gout, 0.0197 for creatinine and 0.0414 for uric acid.
***NPL: nonparametric linkage analysis score, implemented in MERLIN software.
****Deviate method developed by Abecasis et al. (2002) and implemented in MERLIN software.

### The Deviate Method

For quantitative trait linkage analysis, the Deviate method in MERLIN was applied to test for excess sharing among individuals in the same tail of trait distribution for the quantitative trait, without making the normality assumption of the trait distribution, as this method is not sensitive to the assumption of trait distribution. This method is based on the frameworks of Whittemore and Halpern (Whittemore and Halpern (1994) Biometrics 50:118-27) and Kong and Cox (Kong and Cox (1997) Am. J. Hum. Genet. 61: 1179-1188) to define a score function and test the significance of linkage. The quantitative phenotype was subtracted from the population mean, which was based on an epidemiological survey of the same aboriginal tribe for the biochemical measurements. The basic idea is to define score function S(v) by summation of the squared difference of individual phenotypes from the population mean for each founder allele. A Z-mean is used to construct a likelihood ratio test for linkage.

The results showed that quantitative trait locus analysis, involving hyperuricemia subjects and normal subjects, coincidentally mapped to the same location at 114 cM on chromosome 4 (Table 2). For uric acid, the p-value was 0.012, and for creatinine the p-value was 0.005. Ten thousand (10,000) simulations were performed for this analytical method. The empirical P was 0.0414 for uric acid, and 0.0197 for creatinine. There was no evidence of linkage for blood pressures, BMI, triglyceride, and alcohol consumption in this region.

The above results demonstrate that using different analytical approaches and different traits, a gout susceptibility locus is identified on chromosome 4q25 through a genome-wide search on an isolated aboriginal tribe. The probability of type I error is very trivial (less than 1 over 1000 by empirical test), and the significance of linkage increases after adjustment was made for the environmental covariate effect. Interestingly, the marker D4S2623 located at 114 cM of Marshfield genetic map is flanking (only 1.4 cM apart) the Longevity 1 locus (OMIM 606406), marker D4S1564 (Puca et al. (2001) Proc. Nat. Acad. Sci. USA 98:10505-1-508).

### Example 5

### Determination of the Propensity for Gout and Hyperuricemia

The identified genomic region flanked by genome marker pairs of 1) D4S2361 and D4S1644, 2) D4S1647 and D4S2394, or 3) D4S2623 and TAGA006, can be used in a haplotype assay, an assay to determine whether a group of alleles are inherited together as a unit, to ascertain whether a family member of a subject affected with gout (gout subject) has the propensity to become inflicted with gout. The same haplotype analysis involving the same genomic region also can be used to determine whether a family member of a subject affected with hyperuricemia (hyperuricemia subject) has the propensity to become inflicted with hyperuricemia.

This region contains six genomic markers including D4S2361, D4S1647, D4S2623, TAGA006, D4S2394, and D4S1644. (The marker D4S2361 is a tri ATA repeat STRPs and is about 137-173 bp long; the marker D4S1644 is a tetra GATA repeat STRPs and is about 162-222 bp long; the marker D4S1647 is a tetra GATA repeat STRPs and is about 124-164 bp long; the marker D4S2394 is a tri ATA repeat STRPs and is about 232-268 bp long; the marker D4S2623 is a tetra GATA STRPs and is about 225-205 bp long; the marker TAGA006 is a tetra TAGA repeat STRPs and is about 212-256 bp long). The diseased polymorphisms of these markers result in a linkage together to form a haplotype block that presents the maximum-likelihood set of inheritance vectors on gout. This assay can be an invaluable tool in searching for shared segment of distantly related affected individuals.

Thus, the haplotypes of the alleles of these markers of a gout subject are used to determine the propensity for gout in his/her family members. The gout subject's. genomic DNA is prepared from blood with the PureGene DNA Isolation Kit (Gentra system, Minneapolis). The isolated DNA is genotyped with the six STRP markers (D4S2361, D4S1647, D4S2623, TAGA006, D4S2394, and D4S1644) and the haplotype of the six markers is determined. Then, the gout subject's family member is also genotyped to obtain the haplotypes of the same six markers. If the family member's haplotypes and that of the gout subject are identical, then the family member is identified as having the possibility of become affected with gout. The same method can be used with the genome marker D4S2623 and at least one other genome marker selected from D4S2361, D4S1647, TAGA006, D4S2394, and D4S1644. Also, the same method, using the haplotype of a hyperuricemia subject, can be carried out to determine whether the family members of a hyperuricemia subject are likely to have hyperuricemia.

The haplotype assay is a method that can be used to predict and screen gout or hyperuricemia development in family members of subjects having the respective disease. The haplotype assay would help determine whether a gout subject's family member is a gout carrier with the risk of being inflicted with gout or develop hyperuricemia. The assay could also assist in determining whether a hyperuricemia subject's family member is likely to have hyperuricemia. Furthermore, when a gout subject or a hyperuricemia subject and his/her family member have the same disease haplotype, preventive measures can be taken for the family member to avoid or decrease the environmental risk factors such as alcohol drinking, high purine diet etc.

1. An isolated DNA segment, wherein the segment is on chromosome 4 in the genomic region flanked by genome markers D4S2361 and D4S1644.

2. The DNA segment of claim 1, wherein the genomic region is flanked by genome markers D4S1647 and D4S2394.

3. The DNA segment of claim 1, wherein the genomic region is flanked by genome markers D4S2623 and TAGA006.

4. The DNA segment of claim 1, wherein the genomic region is flanked by genome markers D4S2623 and D4S1647.

5. The DNA segment of claim 1, wherein the genomic region is flanked by genome markers D4S2623 and D4S2361.

6. The DNA segment of claim 1, wherein the genomic region is flanked by genome markers D4S2623 and D4S2394.

7. The DNA segment of claim 1, wherein the genomic region is flanked by genome markers D4S2623 and D4S1644.

8. An isolated DNA segment, wherein the segment is in the genomic region of about 90 cM to about 150 cM on chromosome 4.

9. The DNA segment of claim 4, wherein the segment is in the genomic region of about 100 cM to about 140 cM on chromosome 4.

10. The DNA segment of claim 4, wherein the segment is in the genomic region of about 110 cM to about 130 cM on chromosome 4.

11. A method of determining the propensity for gout comprising:
amplifying the DNA segment of claim 1 of a gout subject and his/her family member;
determining the haplotype of genome markers D4S2623 and at least one other genome marker selected from D4S2361, D4S1647, TAGA006, D4S2394, and D4S1644 within the DNA segment of the gout subject and his/her family member;
comparing the haploptye of the gout subject and his family member; and
identifying the family member as having the propensity for gout if the haplotype of the gout subject and that of the family member are identical.

12. The method of claim 11, wherein the DNA segment amplified is the genomic region flanked by genome markers D4S1647 and D4S2394 and wherein the at least one other genome marker is selected from D4S1647, TAGA006, and D4S2394.

13. The method of claim 11, wherein the DNA segment amplified is the genomic region flanked by genome markers D4S2623 and TAGA006 and wherein the at least one other genome marker is TAGA006.

14. A method of determining the propensity for hyperuricemia comprising:
amplifying the DNA segment of claim 1 of a hyperuricemia subject and his/her family member;
determining the haplotype of genome markers D4S2623 and at least one other genome marker selected from D4S2361, D4S1647, TAGA006, D4S2394, and D4S1644 within the DNA segment of the hyperuricemia subject and his/her family member;
comparing the haploptye of the hyperuricemia subject and his family member; and
identifying the family member as having the propensity for hyperuricemia if the haplotype of the hyperuricemia subject and that of the family member are identical.

15. The method of claim 14, wherein the DNA segment amplified is the genomic region flanked by genome markers D4S1647 and D4S2394 and wherein the at least one other genome marker is selected from D4S1647, TAGA006, and D4S2394.

16. The method of claim 14, wherein the DNA segment amplified is the genomic region flanked by genome markers D4S2623 and TAGA006 and wherein the at least one other genome marker is TAGA006.
